(19) United States
(12) Patent Application Publication
(10) Pub. No.: US 2022/0130532 A1
(43) Pub. Date: Apr. 28, 2022

(54) Methods for Managing Digital Work Flow

(71) Applicant: Marc Lemchen, New York, NY (US)

(72) Inventor: Marc Lemchen, New York, NY (US)

(21) Appl. No.: 17/510,729

(22) Filed: Oct. 26, 2021

Related U.S. Application Data
(60) Provisional application No. 63/106,252, filed on Oct. 27, 2020.

(51) Int. Cl. G16H 40/63; G16H 15/00; G16H 50/20

(52) U.S. Cl. CPC G16H 40/63; G16H 50/20; G16H 15/00

(57) ABSTRACT

A method for managing a dental work flow using digital automation. After a remote consultation, the patient is sent a portable scanning wand or device by a dental professional. The patient performs a self intra-oral scan as directed and then transmits the video over a network such as the internet. Before the dental professional can review the transmitted data, a data management module analyzes the transmitted video using a suite of artificial intelligence (AI) tools and automatically prepares an original report or summary which details any likely issues present within the patient's teeth. An additional supplemental report may also be generated by the suite of AI tools which lists any required treatments, any possible elective treatments, and any further treatment options that may be provided responsively to the patient. The dental professional can then form a diagnosis for the patient and then send the diagnosis directly to the patient.

### RELATED APPLICATIONS

The present application is a non-provisional of U.S. provisional application Ser. No. 63/106,252, filed on Oct. 27, 2020, which is incorporated herein by reference in their entirety and to which priority is claimed pursuant to 35 USC 119.

### BACKGROUND

### Field of the Technology

The invention relates to methods for managing a work flow, specifically a work flow related to performing remote dentistry and orthodontic consultations.

### Description of the Prior Art

Telehealth and remote medical consultations, also known as remote patient monitoring (RPM), is a process long in use in which healthcare is provided to a patient by a doctor or other medical professional remotely, i.e. without the traditional in person consultations given to patient's within the doctor's office or other physical setting. The patient typically gives personal medical information to a doctor through a device such as a computer or smart device which is connected to the doctor through a network such as the internet. The doctor may review the received information and then issue a diagnosis or continually provide feedback related to the monitoring of an ongoing treatment plan for the patient. In some instances, an extra external device such as an electrocardiogram machine, thermometer, or other diagnostic device may be sent to the patient within a kit who then performs a diagnostic measurement on themselves or on another individual in their household. The results of the diagnostic measurement are sent to the doctor over the network to aid in their diagnosis or medical supervision. Additionally, with the wide dissemination of cameras and the relative ease and availability of video calls, doctors increasingly perform visual examine patients remotely.

Remote patient monitoring and telehealth medical practice has only accelerated over the last two years where in person examinations have become increasingly inconvenient or undesired in view of the health risks associated with the COVID-19 pandemic. This trend is likely to continue in the foreseeable future as IT and network technology only become more powerful and instrumental to everyday life.

While remote patient monitoring or remote telehealth consultations lend themselves well to general examinations, post operative monitoring, and even prenatal visits, such procedures are largely still incompatible or ill suited for services related to dentistry and orthodontics. This is largely because in order to properly perform an oral examination, the dentist or orthodontist must view the inside of the patient's mouth, something which is not easily accomplished using a commercial web camera. Furthermore, to the extent that an oral examination can be performed remotely, the quality of the information that the dentist or orthodontist receives would be less then ideal or helpful given the current constraints of RPM or telehealth examinations.

What is needed is a method for performing oral examinations remotely which will provide the supervising dentist or orthodontist all the data and information necessary to provide the patient with a diagnosis and/or a treatment regimen. The method should further compensate for a lack of in person analysis by automatically providing a preliminary report on the scan performed by the patient which further assists the dentist or orthodontist in making a diagnosis.

### BRIEF SUMMARY

The current invention provides a method for managing a digital work flow related to performing intra-oral examinations. The method includes performing an intra-oral scan by a patient, transmitting data from the intra-oral scan to an automated system that is in the control of a dental professional, and automatically analyzing the transmitted data for potential dental or orthodontic issues in the patient using a suite of artificial intelligence (AI) tools disposed within the automated system. Next, a report is generated which contains at least one result of the analysis previously performed on the transmitted data. The original data transmitted by the patient and the generated report are then provided to the dental professional who in turn provides the patient with a diagnosis or treatment plan.

In one embodiment, the method also includes automatically capturing a set of data that is related to the patient by the automated system. Such data capturing may specifically including recording the moment when the patient has completed a review of a set of instructions that were previously provided to the patient.

In another embodiment, the method further includes completing an initial contact between the patient and the dental professional and then sending the patient an intra-oral scanner.

In yet another embodiment, the step of performing the intra-oral scan by the patient specifically includes attaching an intra-oral scanner to a smart device.

In another embodiment, the step of transmitting data from the intra-oral scan to the automated system that is in control of the dental professional includes uploading the intra-oral scan data to a dedicated portion of the automated system.

In a further embodiment, automatically analyzing the transmitted data for potential dental or orthodontic issues in the patient using the suite of artificial intelligence (AI) tools disposed within the automated system specifically involves detecting possible cavities, periodontal issues, malocclusions, or any restorative work in place related to crowns, bridges, implants, or veneers within the patient.

In one particular embodiment, the step of providing the transmitted data and the generated report to the dental professional explicitly includes simultaneously providing the transmitted data and the generated report to the dental professional only after the report has been generated by automated system.

In one particular embodiment, the method further includes generating a supplemental report that contains at least one treatment option that is directed to the patient. The treatment option is directly based on the results of the analysis performed on the transmitted data. Both the first generated report and the supplemental report are preferably generated by the suite of AI tools that are disposed within the automated system. Additionally, the supplemental report is also provided to the dental professional at the same time as any other data or analysis provided by the automated system. In one particular related embodiment, the dental professional may selectively determine when or whether the supplemental report is generated by the automated system.

In another embodiment, the method may also include revising the diagnosis or treatment plan formed by the dental professional with a medical history related to the patient.

Additionally, in another particular embodiment, the patient is provided with a set of instructions for how to perform the intra-oral scan with an intra-oral scanner that has been previously sent to the patient.

The current invention also provides a method for using a digital remote dentistry work flow. The method includes performing an initial remote examination of a patient by a dental professional who then sends the patient an intra-oral scanning device. The patient then performs an intra-oral scan and transmits the data from the intra-oral scan to an automated system associated with the dental professional. After receiving a report generated by the automated system which includes at least one result of an analysis performed on the transmitted data, the dental professional forms a diagnosis or treatment plan that is based at least in part on the generated report who then provides the patient with the diagnosis or treatment plan.

In one embodiment, the patient is also provided with a set of instructions for how to perform the intra-oral scan with the intra-oral scanning device that was previously sent to the patient.

In another embodiment, a supplemental report generated by the automated system is received by the dental professional, the generated supplemental report itself having at least one treatment option directed to the patient, wherein the at least one treatment option is further based on the results of the analysis previously performed on the transmitted data.

In yet another embodiment, transmitting data from the intra-oral scan to the automated system of the dental professional specifically includes the patient uploading the intra-oral scan data to a dedicated portion of the automated system.

In a further embodiment, forming the diagnosis or treatment plan by the dental professional that is based at least in part on the generated report explicitly includes revising the diagnosis or treatment plan with a medical history that is related to the patient.

In one particular embodiment, the step of forming the diagnosis or treatment plan by the dental professional also includes incorporating the generated supplemental report which includes at least one treatment option that is directed to the patient.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a flow chart illustrating a preferred embodiment of managing of a digital work flow of the current invention.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

The current invention solves the problem of performing a remote dental scan presented by the prior art by providing a method for managing a dental work flow using digital automation. Specifically, a dental work flow method begins with an initial patient contact which can be any traditional form of contact such as phone calls, emails, or direct patient registration on the dentist's or orthodontist's website for dental treatment which can then be followed up by a teledentistry consultation through a subsequent phone or video call. The patient's information including general contact information and any other pre-treatment information given by the patient is recorded in an automated system which is in the control of the dentist or orthodontist. The patient is then sent a portable and wireless scanning wand or device by the dentist or orthodontist. In one embodiment, the wireless scanning wand may be a standalone component or device, or alternatively, the wireless scanning wand may be a detachable modification or attachment for the patient's smartphone or other smart device. The wireless scanning wand arrives sterilized and is packaged with scanning instructions. The automated system further notes when the patient was sent the wireless scanning wand so as to track the patient's progress through the overall treatment process.

The wireless scanning wand is further accompanied with an instructive video which is either included with the wireless scanning wand itself or which is more preferably sent to the patient contemporaneously via a link or well-known video format. The wireless scanning wand is accurate enough to provide a reliable and competent scan of the patient's oral cavity. In one particular embodiment, the automated system tracks the progress of the patient as they review the provided tutorials or instructional material. For example, the automated system may mark when the patient has completed viewing a video that was initially provided to the patient through a link sent by the dentist or orthodontist.

After reviewing the written and/or video instructions, the patient performs a self-scan of their teeth and gums as directed. Alternatively, when the patient is a child or a pet, an adult or other designated person may review the instructions and then scan the child or pet as directed in order to look for dental health issues.

After scanning is complete, the patient transmits the video to the supervising dentist or orthodontist over a network such as the internet. Before the dentist or orthodontist can review the transmitted video however, a data management module which is part of the automated system that is stored either on the dental professional's computer or on the network first analyzes the transmitted video using a suite of artificial intelligence (AI) tools or algorithms and automatically prepares an original report or summary which details any likely cavities, periodontal issues, any restorative work in place related to crowns, bridges, implants, or veneers, and malocclusions. The suite of AI tools may exist within a single software program or it may be distributed across the Cloud or other network which is connected to the dentist or orthodontist. The supervising dentist or orthodontist then receives the generated report and the original transmitted scan from the patient at the same time. The dentist or orthodontist may review the possible issues initially flagged or noted by the report so as to have them top of mind when they specifically review the original transmitted scan in greater or focused detail. The dentist or orthodontist may disregard the issues noted within the report if it is not supported by their own analysis of the transmitted dental scan, or alternatively, confirm the presence of said issues after completing their analysis of the transmitted scan.

In another embodiment, an additional supplemental report is generated by the suite of AI tools which lists any required treatments, any possible elective treatments, and any further treatment options that may be provided responsively to the patient. In other words, the supplemental report is directed to evidence-based options that are directly based on the previously derived information from the patient's intraoral scan. The automated system then automatically makes referrals to the dentist or orthodontist based upon predetermined criteria previously selected by the dentist or orthodontist.

The dentist or orthodontist may then form a diagnosis for the patient and then send the diagnosis directly by either email, a phone call, text, or through a subsequent teleconference or video call. An optional tele dentistry consultation may also be had between the patient and the dentist or orthodontist to discuss the findings of the both the AI generated reports and the opinion of the dentist or orthodontist. In an additional optional step, the patient may transmit their medical records to the dentist or orthodontist in order to take into account any possible constraints based upon the patient's prior medical history. The patient then follows the diagnoses or treatment plan recommended by the dentist or orthodontist.

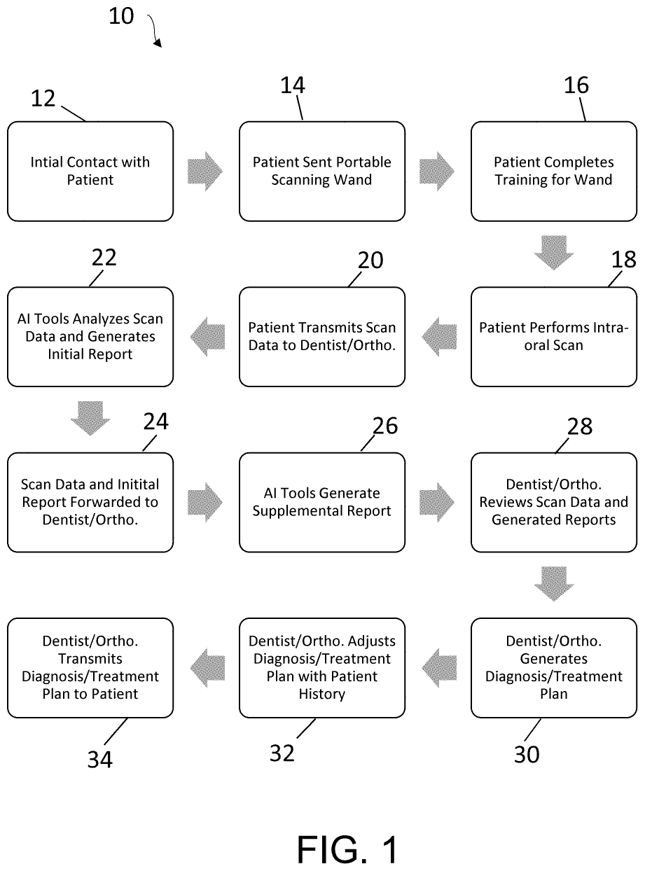

Turn now to a specific embodiment of the current method for managing a digital work flow shown in FIG. 1 which is denoted generally by reference numeral 10. The method 10 begins in step 12 where initial contact between the patient and the supervising dentist or orthodontist is made through any conventional means now known or later devised including but not limited to phone calls, emails, or video conference calls. Each time the patient and dentist or orthodontist communicate with each other, it is tracked and recorded by an automated system, thereby creating a communication log or history which may be referred to later if needed. The automated system operates principally as a software platform that is maintained and controlled by the dentist or orthodontist and which operates the method 10 of the current invention. Also in step 12, the automated system records or captures any initial data related to the patient which may contain general contact information, any previously existing medical or dental records, or initial notes drafted by the dentist or orthodontist.

Next in step 14, the dentist or orthodontist sends the patient a portable, wireless scanning wand. The scanning wand arrives in pre-packaged, sterile packaging that is safe for human use. The scanning wand in one embodiment is a stand alone device which may be used immediately upon arrival, however in a separate embodiment, in step 14 the patient must first connect or pair the scanning wand to their smart device as well as download and install any applications to their smart device which may be necessary to operate or use the scanning wand itself. Regardless of the type of scanning wand used, delivery of the scanning wand device to the patient may be recorded by the automated system through standard mail tracking or commonly available delivery confirmation means.

The scanning wand may be packaged with a set of written instructions for using the scanning wand, or alternatively, the name of a website or a link may be provided which directs the patient to an online set of instructions or an instructional video. The patient then reviews the training materials in step 16 so as to understand and better prepare themselves for performing an intra-oral dental scan. In the instance where the instructional materials are online or otherwise connected to a network, the automated system may record when the patient has completed reviewing the instructional materials, thereby indicating to the dentist or orthodontist that the patient is preparing to use the scanner device and that scan data is likely forthcoming. The patient then performs the intra-oral scan of their oral cavity according to the provided instructions in step 18.

Once the intra-oral scan is complete, the patient transmits the scan data to the dentist or orthodontist in step 20. In one preferred embodiment, the patient transmits the scan data to the dentist or orthodontist via a file which is sent as an attachment in an email. However, other modes of transmission may be used without departing from the original spirit and scope of the invention. For example, the patient may directly upload the scan data directly to the dentist's or orthodontist's website or other dedicated online portal which is connected to or is part of the automated system. Regardless, all incoming scan data transmitted by the patient is intercepted and logged by the automated system.

Before the dentist or orthodontist is permitted to directly review the transmitted scan data, the automated system in step 22 performs a plurality of analysis steps or sub-routines using one or more artificial intelligence (AI) tools that are well known in the art. Each of the one or more AI tools are principally configured to review and analyze image and video data, and more specifically, each of the one or more AI tools are configured to review the transmitted scan data for dental or orthodontic issues such as but not limited to cavities, periodontal issues, malocclusions, or any restorative work in place related to crowns, bridges, implants, or veneers. Once analysis has been completed, a summary or report of the results obtained from the AI tools is generated by the automated system. The automated system records or saves the generated report so that it is permanently associated with the patient who originally transmitted the scan data. The automated system then sends or makes available to the dentist or orthodontist both the generated report and the original scan data transmitted by the patient in step 24.

In addition to generating an automated primary report which flags or highlights possible dental or orthodontic issues, the automated system in step 26 may further automatically generate a supplemental report which produces a series of recommended treatments or procedures related to the patient which is directly based on the possible issues previously flagged within the primary generated report. The same suite of AI tools used to generate the primary report may also be used to generate the second or supplemental report, or alternatively a second set of one or more different or additional AI tools may be used to exclusively generate the supplemental report. The generated supplemental report is further forwarded or made available to the dentist or orthodontist who may then consider it along with the first general report and the original scan data transmitted by the patient. In one embodiment, step 26 is optional or is actively selectable by the dentist or orthodontist. In other words, the dentist or orthodontist may optionally request the automated system to generate the secondary report when additional recommendations or treatment options related to the patient's current condition are desired. Alternatively, the dentist or orthodontist may forego the second or supplementary report and skip step 26 completely when no treatment recommendations are needed.

Once the automated system releases the scan data transmitted to the automated system by the patient and any previously generated reports to the dentist or orthodontist, the dentist or orthodontist reviews and analyzes the scan data and the generated reports in step 28. The dentist or orthodontist then synthesizes the scan data and the generated reports with their own observations and knowledge in step 30 to formulate or develop a final diagnosis and/or a future treatment plan or regimen for the patient.

In optional step 32, the dentist or orthodontist may revise or supplement their diagnosis or treatment plan with the patient's prior medical history as provided by the patient. The patient may provide the dentist or orthodontist with their medical record upon the request of the dentist or orthodontist, or alternatively, the patient may have already previously provided their medical history to the automated system when initial contact was begun under step 12.

Finally, in step 34, the dentist or orthodontist transmits or communicates their diagnosis and/or treatment plan to the patient. The dentist or orthodontist may send the patient their results via text, email, or through a phone call, however in a preferred embodiment the dentist or orthodontist discusses the results of the intra-oral scan and any possible treatment options through a video call so as to more closely replicate a traditional face to face consultation. Future appointments for in person consultations or procedures may then be scheduled as needed.

Using the digital management work flow of the current method 10, dentists and orthodontists are given the ability to remotely consult their patients and provide an accurate diagnosis of any potential issues which may have gone otherwise unnoticed if the patient was unable to come in for a traditional in person examination. Using AI assisted imaging and analysis tools allows the dental professional to not only provide a targeted or focused examination of the patient, but it also further compensates for the dental professional's inability to perform an in-person examination. The net result therefore is a dental examination of the patient which is as good or better than a traditional in-person consultation.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

I claim:

1. A method for managing a digital work flow related to performing intra-oral examinations, the method comprising:
performing an intra-oral scan by a patient;
transmitting data from the intra-oral scan to an automated system in control of a dental professional;
automatically analyzing the transmitted data for potential dental or orthodontic issues in the patient using a suite of artificial intelligence (AI) tools disposed within the automated system;
generating a report containing at least one result of the analysis performed on the transmitted data;
providing the transmitted data and the generated report to the dental professional; and
providing the patient with a diagnosis or treatment plan by the dental professional.

2. The method of claim 1 further comprising automatically capturing a set of data related to the patient by the automated system.

3. The method of claim 1 further comprising:
completing an initial contact between the patient and the dental professional; and
sending the patient an intra-oral scanner.

4. The method of claim 1 wherein performing the intra-oral scan by the patient comprises attaching an intra-oral scanner to a smart device.

5. The method of claim 1 wherein transmitting data from the intra-oral scan to the automated system in control of the dental professional comprises uploading the intra-oral scan data to a dedicated portion of the automated system.

6. The method of claim 1 wherein automatically analyzing the transmitted data for potential dental or orthodontic issues in the patient using the suite of artificial intelligence (AI) tools disposed within the automated system comprises detecting possible cavities, periodontal issues, malocclusions, or any restorative work in place related to crowns, bridges, implants, or veneers within the patient.

7. The method of claim 1 wherein providing the transmitted data and the generated report to the dental professional comprises simultaneously providing the transmitted data and the generated report to the dental professional only after the report has been generated by automated system.

8. The method of claim 1 further comprising generating a supplemental report containing at least one treatment option directed to the patient, wherein the at least one treatment option is based on the at least one result of the analysis performed on the transmitted data.

9. The method of claim 8 wherein the generated report containing at least one result of the analysis performed on the transmitted data and the generated supplemental report containing at least one treatment option directed to the patient are generated by the suite of AI tools disposed within the automated system.

10. The method of claim 8 wherein providing the transmitted data and the generated report to the dental professional comprises providing the generated supplemental report to the dental professional.

11. The method of claim 1 further comprising revising the diagnosis or treatment plan with a medical history related to the patient.

12. The method of claim 1 further comprising providing the patient a set of instructions for how to perform the intra-oral scan with an intra-oral scanner provided to the patient.

13. The method of claim 2 wherein automatically capturing the set of data related to the patient by the automated system comprises recording when the patient has completed a review of a set of instructions previously provided to the patient.

14. The method of claim 8 further comprising the dental professional selectively determining when the supplemental report is generated by the automated system.

15. A method for using a digital remote dentistry work flow comprising:
performing an initial remote examination of a patient by a dental professional;
sending the patient an intra-oral scanning device;
performing an intra-oral scan by the patient;
transmitting data from the intra-oral scan to an automated system of the dental professional;
receiving a report generated by the automated system by the dental professional, the generated report comprising at least one result of an analysis performed on the transmitted data;
forming a diagnosis or treatment plan by the dental professional based at least in part on the generated report; and
providing the patient with the diagnosis or treatment plan.

16. The method of claim 15 further comprising providing the patient a set of instructions for how to perform the intra-oral scan with the intra-oral scanning device sent to the patient.

17. The method of claim 15 further comprising receiving a supplemental report generated by the automated system by the dental professional, the generated supplemental report comprising at least one treatment option directed to the patient, wherein the at least one treatment option is based on the at least one result of the analysis performed on the transmitted data.

18. The method of claim 15 wherein transmitting data from the intra-oral scan to the automated system of the dental professional comprises uploading the intra-oral scan data to a dedicated portion of the automated system by the patient.

19. The method of claim 15 wherein forming the diagnosis or treatment plan by the dental professional based at least in part on the generated report comprises revising the diagnosis or treatment plan with a medical history related to the patient.

20. The method of claim 17 wherein forming the diagnosis or treatment plan by the dental professional based at least in part on the generated report comprises incorporating the generated supplemental report comprising at least one treatment option directed to the patient.
